SEQUENCE LISTING (16 sequences)

SEQ ID NO: 1 - protein, 247 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARVDAMYYPYFDYWGQGTLVTVSS

SEQ ID NO: 2 - protein, 250 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPSTFGPGTKVDIKSGILGTTAASGSSGGSSSGAEVQLVQSGAEVKKPGESLKISCKGSGYSFTSYWIGWVRQMPGKGLEWMGIIYPGDSDTRYSPSFQGQVTISADKSISTAYLQWSSLKASDTAVYYCARYDSGGLYYYDFDYWGQGTLVTVSS

SEQ ID NO: 3 - protein, 245 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARNYELDYFDYWGQGTLVTVSS

SEQ ID NO: 4 - protein, 251 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARYDYGSDSPLHRSFDYWGQGTLVTVSS

SEQ ID NO: 5 - protein, 245 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDGYYSDFDYWGQGTLVTVSS

SEQ ID NO: 6 - protein, 250 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARYDYGSDAGLTYFDYWGQGTLVTVSS

SEQ ID NO: 7 - protein, 245 aa, unidentified
EIVLTQSPGTLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDYLYSDFDYWGQGTLVTVSS

SEQ ID NO: 8 - protein, 251 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGSGYSGYTAADDFDYWGQGTLVTVSS

SEQ ID NO: 9 - protein, 250 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKSGILGTTAASGSSGGSSSGAQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARYYDLYDSYPEYFDYWGQGTLVTVSS

SEQ ID NO: 10 - protein, 104 aa, unidentified
ARVDQTPQTITKETGESLTINCVVRDTACALVRTFWYRKKSGSTNEESISKGGRYVETVNRGSKSFSLRINDLTVEDSGTYRCKVFGWCRMRDVYGGGTVVTVN

SEQ ID NO: 11 - protein, 109 aa, unidentified
ARVDQTPQTITKETGESLTINCVLRDASYALGSTCWYRKKSGSTNEESISIGGRYVETVNSGSKSFSLRINDLTVEDSGTYRCGVDSWCPPVCSWAYAGCGDGTAVTVN

SEQ ID NO: 12 - protein, 110 aa, unidentified
ARVDQTPQTITKETGESLTINCVLRDSNCALSSTYWYRKKSGSTNEESISKGGRYVETVNSGSKSFSLRINDLTVEDSGTYRCRLFVSWLSDYDCATYYVSGGGTVVTVN

SEQ ID NO: 13 - protein, 104 aa, unidentified
ARVDQTPLTITKETGESLTINCVLRDSNCALSSTYWYRKSSGSTNEEIISKGGRYVETVNSGSNSFSLRINDLTVEDSGTYRCKVFGWCRMRDVYGGGTVVTVN

SEQ ID NO: 14 - protein, 104 aa, unidentified
ARVDQTPQTITKETGESLTINCVVRDSACALVRTFWYRKKSGSTNEESISKGGRYVETVNSGSKSFSLRINDLTVEDSGTYRCKVFGWCRMRDVYGGGTVVTVN

SEQ ID NO: 15 - protein, 15 aa, unidentified
GGGGSGGGGSGGGGS

SEQ ID NO: 16 - protein, 10 aa, unidentified
GGGGSGGGGS

(19) United States
(12) Patent Application Publication
(10) Pub. No.: US 2026/0049144 A1
(43) Pub. Date: Feb. 19, 2026

(54) ANTI-PD-L1 MULTIPARATOPIC ANTIBODY CONSTRUCTS AND USES THEREOF

(71) Applicants: The Johns Hopkins University, Baltimore, MD (US); University of Maryland, Baltimore, Baltimore, MD (US)

(72) Inventors: Jamie Spangler, Baltimore, MD (US); Seth Ludwig, Baltimore, MD (US); Angela Zhu, Baltimore, MD (US); Rakeeb Kureshi, Baltimore, MD (US); Helen Dooley, Baltimore, MD (US)

(21) Appl. No.: 19/103,101

(22) PCT Filed: Aug. 10, 2023
(86) PCT No.: PCT/US2023/072013

§ 371 Date: Feb. 11, 2025

Related U.S. Application Data
(60) Provisional application No. 63/397,643, filed on Aug. 12, 2022.

(51) Int. Cl. C07K 16/28; A61K 39/00; A61P 35/00

(52) U.S. Cl. CPC C07K 16/2827; A61P 35/00; A61K 2039/505; C07K 2317/33; C07K 2317/569; C07K 2317/76; C07K 2317/92

(57) ABSTRACT

Provided herein are anti-PD-L1 multiparatopic antibody constructs that include a plurality of antigen-binding fragments, wherein a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope.

### CROSS-REFERENCE TO RELATED APPLICATIONS

This application claims priority to U.S. Provisional Patent Application No. 63/397,643, filed on Aug. 12, 2022, which is incorporated herein by reference in its entirety.

### FEDERALLY SPONSORED RESEARCH OR DEVELOPMENT

This invention was made with government support under grant nos. CA236616, CA249381 and EB024495, awarded by the National Institutes of Health, and under grant no. 2143160, awarded by the National Science Foundation. The government has certain rights in the invention.

### SEQUENCE LISTING

This application contains a Sequence Listing that has been submitted electronically as an XML file named “44807-0437WO1_ST26_SL.XML.” The XML file, created on Aug. 10, 2023, is 17,441 bytes in size. The material in the XML file is hereby incorporated by reference in its entirety.

### TECHNICAL FIELD

The present disclosure relates to the field of biotechnology, and more specifically, to anti-PD-L1 multiparatopic antibody constructs and uses thereof.

### BACKGROUND

Programmed death-ligand 1 (PD-L1) is a member of the B7 family of immunoregulatory ligands that play a pivotal role in regulation of the cellular and humoral immune responses. PD-L1 is expressed in both lymphoid and non-lymphoid tissue, where it is involved in suppression of immune activity through activation of the co-inhibitory receptor programmed cell death protein-1 (PD-1), which is expressed primarily on T cells, but also some natural killer (NK) cells and monocytes. Immunosuppressive pathways (also called immune checkpoint pathways) such as the PD-1/PD-L1 axis are essential for maintaining healthy immune regulation. However, these pathways are frequently exploited by cancer cells and immune cells in the tumor microenvironment to evade immune recognition. Indeed, PD-L1 expression is observed in various forms of cancer, both constitutively and as a feedback response to inflammatory signals, resulting in inhibition of tumor specific T cell responses. To counteract this effect, monoclonal antibodies known as immune checkpoint inhibitors (ICIs) have been developed to target immune checkpoint proteins such as PD-L1. ICIs that disrupt the PD-1/PD-L1 interaction have shown success in treating a variety of cancers, including 3 FDA-approved anti-PD-L1 antibodies: atezolizumab, durvalumab, and avelumab. However, the immunological mechanisms by which anti-PD-L1 therapies function are complex and still only partially understood.

Blockade of the PD-1/PD-L1 pathway restores functionality to chronically exhausted CD8+ T cells, suggesting that tumor-associated PD-L1 primarily inhibits proliferation and effector functions of activated tumor-specific T cells. Upregulation of PD-L1 by tumor cells is often associated with adaptive resistance to endogenous tumor-specific immune responses, in particular the secretion of interferon-γ (IFN-γ) as well as inflammatory cytokines. Although high-level expression of PD-L1 in tumor biopsies has generally been used as a criterion to guide anti-PD-L1 treatment, it has become increasingly clear that PD-L1 expression on infiltrating immune cells, including myeloid cells and T cells, can in many cases be sufficient to predict therapeutic efficacy. Some data suggest that PD-L1 expression on antigen-presenting cells may play a vital role in determining the efficacy of anti-PD-L1 therapy, whereas PD-L1 on tumor cells and tumor infiltrating lymphocytes is irrelevant, highlighting the continued uncertainty on the underlying mechanisms of PD-L1-targeted ICIs. Despite incomplete understanding of the immune effects of anti-PD-L1 antibodies, these drugs have been shown to achieve complete and durable cures in some patients, which has led to their approval across cancer types, including non-small cell lung cancer (NSCLC) and Merkel cell carcinoma. Unfortunately, patient response rates to anti-PD-L1 therapies remain disappointingly low and most tumor regressions are only partial, necessitating the development of new mechanistic strategies to enhance the efficacy of immune checkpoint protein targeted therapies.

### SUMMARY

Provided herein are anti-PD-L1 multiparatopic antibody constructs comprising a plurality of antigen-binding fragments, wherein a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope. In some embodiments, an antigen-binding fragment of the plurality of antigen-binding fragments comprises a variable domain, wherein the variable domain binds specifically to a third PD-L1 epitope. In some embodiments, the anti-PD-L1 multiparatopic antibody construct comprises a human immunoglobulin G1 (IgG) antibody. In some embodiments, the human IgG antibody comprises atezolizumab.

In some embodiments, the anti-PD-L1 multiparatopic antibody construct further comprises (i) a first polypeptide comprising a light chain, and (ii) a second polypeptide comprising a heavy chain. In some embodiments, the first antigen-binding fragment and/or the second antigen-binding fragment is conjugated to the first polypeptide. In some embodiments, the first antigen-binding fragment and/or the second antigen-binding fragment is N-terminally positioned relative to the light chain. In some embodiments, the first antigen-binding fragment and/or the second antigen-binding fragment is C-terminally positioned relative to the light chain. In some embodiments, the first antigen-binding fragment and/or the second antigen-binding fragment is conjugated to the second polypeptide. In some embodiments, the first antigen-binding fragment and/or the second antigen-binding fragment is N-terminally positioned relative to the heavy chain. In some embodiments, the first antigen-binding fragment and/or the second antigen-binding fragment is C-terminally positioned relative to the heavy chain.

In some embodiments, the first polypeptide comprises an engineered light chain constant region, and wherein the second polypeptide comprises an engineered heavy chain constant region, wherein the engineered light chain constant region and the engineered heavy chain constant region preferentially associate with each other as compared to non-engineered light and heavy chain constant regions. In some embodiments, the engineered heavy chain constant region comprises a mutation. In some embodiments, the mutation is a N297A mutation.

In some embodiments, the anti-PD-L1 multiparatopic antibody construct further comprises a linker sequence. In some embodiments, the linker sequence comprises a (G₄S)₃ or a (G₄S)₂ linker. In some embodiments, the linker sequence is positioned between the first polypeptide and the first antigen-binding fragment and/or the second antigen-binding fragment. In some embodiments, the linker sequence is positioned between the second polypeptide and the first antigen-binding fragment and/or the second antigen-binding fragment.

In some embodiments, the first antigen-binding fragment and/or the second antigen-binding fragment comprises a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the first antigen-binding fragment and/or the second antigen-binding fragment comprises a sequence of SEQ ID NO: 9, SEQ ID NO: 10, or SEQ ID NO: 11. In some embodiments, the plurality of antigen-binding fragments comprise two or more sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the plurality of antigen-binding fragments comprise two or more sequences selected from the group consisting of: SEQ ID NO: 9, SEQ ID NO: 10, or SEQ ID NO: 11.

In some embodiments, the antigen-binding fragment comprises an antibody. In some embodiments, the antigen-binding fragment comprises a scFv. In some embodiments, the antigen-binding fragment comprises a VNAR.

Also provided herein are nucleic acids comprising sequences that encode polypeptides making up any one of the anti-PD-L1 multiparatopic antibody constructs described herein.

Also provided herein are vectors comprising any one of the nucleic acids described herein.

Also provided herein are cell comprising any one of the nucleic acids or any one of the vectors described herein.

Also provided herein are pharmaceutical compositions comprising any one of the anti-PD-L1 multiparatopic antibody constructs, any one of the nucleic acids, any one of the vectors, or any one of the cells described herein, and a pharmaceutically acceptable carrier.

Also provided herein are kits comprising any one of the pharmaceutical compositions described herein.

Also provided herein are methods of producing an anti-PD-L1 multiparatopic antibody construct, the method comprising: (a) culturing any one of the cells described herein in a liquid culture medium under conditions sufficient to produce the anti-PD-L1 multiparatopic antibody construct; and (b) recovering the anti-PD-L1 multiparatopic antibody construct from the cell and/or liquid culture medium. In some embodiments, the method further comprises isolating the recovered anti-PD-L1 multiparatopic antibody construct. In some embodiments, the method further comprises formulating the isolated anti-PD-L1 multiparatopic antibody construct into a pharmaceutical composition.

Also provided herein are methods of treating a disease in a subject in need thereof, the method comprising administering a therapeutically effective amount of any one of the anti-PD-L1 multiparatopic antibody constructs or any one of the pharmaceutical compositions described herein to the subject. In some embodiments, the subject is a human. In some embodiments, the disease is a cancer. In some embodiments, the cancer is a bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, prostate cancer, or combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1E show discovery of novel PD-L1-targeted antibodies. FIG. 1A shows an exemplary schematic representation of receptor trafficking following treatment with a biparatopic antibody that targets two non-competitive epitopes on PD-L1. FIGS. 1B-1C show progression of PD-L1 binding (1000 nM) over four rounds of selections against 2 yeast surface-displayed libraries: (FIG. 1B) a synthetic scFv library; and (FIG. 1C) a VNAR library derived from immunization of sharks with the PD-L1 extracellular domain. FIGS. 1D-1E show on-yeast PD-L1 binding titrations of individual clones isolated from: (FIG. 1D) the synthetic scFv library; and (FIG. 1E) the immunization derived VNAR library, as measured by flow cytometry analysis.

FIG. 2A-2E show the design of exemplary multiparatopic antibodies targeting PD-L1. FIG. 2A shows equilibrium binding affinities of two scFv (atezolizumab and D12) and two VNAR (B8 and A9) fragments against immobilized PD-L1, as measured by bio-layer interferometry. FIGS. 2B-2D show cross-competition analysis of PD-L1 binding between two scFvs (atezolizumab and D12) and two VNARs (B8 and A9). Charts depict bio-layer interferometry analysis of equilibrium binding of an immobilized antibody fragment (either (FIG. 2B) atezolizumab, (FIG. 2C) D12, and (FIG. 2D) B8) to saturating amounts of PD-L1 ((FIG. 2B, 2D) 100 nM, (FIG. 2C) 1000 nM) in the presence of varying concentrations of the indicated soluble competitor. FIG. 2E shows exemplary schematic depictions of bi- and tri-paratopic antibody fusion proteins. These constructs are based on the atezolizumab human IgG1 antibody, and include either N or C terminal fusions to the heavy and/or light chains, or asymmetric knobs-into-holes Fc mutations.

FIGS. 3A-3B show topology and epitope dependence of antibody-mediated PD-L1 downregulation. MDA-MB-231 human breast cancer cells were treated with the specified (FIG. 3A) biparatopic or (FIG. 3B) triparatopic antibody for 12 hours, and then surface PD-L1 was quantified by flow cytometry. All values were normalized to an untreated control (black). Error bars represent the standard deviation from three replicates. Statistical significance was determined by one-way ANOVA.

FIGS. 4A-4G show antibody-induced clustering and internalization of PD-L1. FIG. 4A shows quantification of PD-L1 receptor expression on 5 human cancer cell lines via flow cytometry. FIG. 4B shows the indicated cells were treated with atezolizumab (red) or the multiparatopic antibody TS1521 (purple) for 12 hours, and then surface PD-L1 was quantified by flow cytometry. Data were normalized relative to an untreated control (black). Error bars represent s.d. (n=3), and significance was determined by one-way ANOVA. FIG. 4C shows H2444 cells were exposed to atezolizumab (red), TS1521 (purple) or were left untreated (black) for varying time periods. Surface receptor levels were quantified by flow cytometry. Error bars represent s.d. (n=3). FIG. 4D shows confocal imaging of PD-L1 on live H2444 human lung cancer cells treated with Alexa Fluor 488-conjugated atezolizumab or TS1521 at different time points. The scale bars measure 14 μm. FIG. 4E shows representative regions of interest (ROIs) sampled from super resolution (SR) imaging data of H2444 cells treated with Alexa Fluor 647-conjugated atezolizumab or TS1521 for 12 hr. The scale bars measure 1000 nm. The dots on these images are Gaussian representations of the point spread function for each localization detected. FIG. 4F shows cluster boundaries (in magenta) found by DBSCAN superposed on top of the SR localization locations (black points) from the SR images shown in FIG. 4E. The green boxes define the ROI boundaries. The points that fall outside the magenta boundaries are counted as not clustered. DBSCAN was run with parameters epsilon=100 nm, minimum number of localizations to form a cluster=10. FIG. 4G shows a box plot depicting the percent of clustered localizations per ROI for H2444 cells treated with atezolizumab and TS1521, determined as shown in FIG. 4F. In this plot, the middle bars indicate the median, the edges of the boxes extend from the 25th to the 75th percentiles of the data, and the whiskers reach to the most extreme data. Significance was determined by two-sample Kolmogorov-Smirnov goodness-of-fit hypothesis test.

FIGS. 5A-5E show antibody-mediated effects on PD-L1 trafficking and subcellular localization. FIG. 5A shows surface PD-L1 expression time course in H2444 cells treated with TS1521 (purple) or untreated control (black) in either the presence (closed circles) or absence (open circles) of the recycling inhibitor monensin. Error bars represent s.d. (n=3). FIG. 5B shows H2444 cells were pulsed with Alexa Fluor 488-conjugated atezolizumab (red) or TS1521 (purple) for 2 hr at 37° C., followed by quenching of surface signal at 4° C. Cells were then chased at 37° C. in the continued presence of quenching antibody for the indicated time period, such that further decreases in signal would reflect recycling of internalized PD-L1. Data are plotted normalized to surface PD-L1 signaling following the initial quenching reaction. Error bars represent s.d. (n=3). FIG. 5C shows H2444 cells were treated with either atezolizumab (red) or TS1521 (purple) conjugated to a pH-sensitive fluorescent dye for the indicated amount of time. Error bars represent s.d. (n=3). Significance was determined by two-way ANOVA. FIG. 5D shows confocal microscopy imaging of H2444 cells treated with Alexa Fluor 488-conjugated atezolizumab or TS1521 (green) for 13 hr, then stained for endosomes (blue, EEA1) and lysosomes (red, LAMP-1) to assess colocalization. The scale bars measure 28 μm. FIG. 5E shows fraction of endosomes and lysosomes from images presented in FIG. 5D that are associated with atezolizumab (red) or TS1521 (purple).

FIGS. 6A-6B show enhancement of immune cell activation following antibody-mediated downregulation of PD-L1. FIG. 6A shows activation readouts from a commercial TCR signaling reporter assay. Atezolizumab (red, left) or TS1521 (purple, right) was added to PD-L1-expressing antigen-presenting cells (APCs) for 2 hr to allow for downregulation to occur. Subsequently, the antibodies were maintained in some samples (solid lines), while in others the antibody was washed away (dotted lines). The APCs were then incubated with effector (Jurkat) cells, and NFAT-induced luminescence was used as a readout for TCR signaling, reflecting the extent of PD-1/PD-L1 blockade. Error bars represent s.d. (n=3). FIG. 6B shows activation readouts from a primary human T cell functional assay. PBMCs from two independent human donors with T cells specific for HCV peptides were first treated with either atezolizumab (red) or TS1521 (purple), then stimulated with peptide in either the continued presence of the antibody (closed circles), or with the antibody washed off (wash, open circles). Activation was quantified by ELISpot analysis of interferon-γ secretion. The signal reported is normalized to the untreated control, and combines data from two identical experiments. Error bars represent s.d. Significance was determined by one-way ANOVA.

FIGS. 7A-7C show in vivo pharmacodynamics of PD-L1-targeted multiparatopic antibody. FIG. 7A shows antibody localization and persistence in MDA-MB-231 tumors in NSG mice after i.v. (tail vein) injection of 10 mg/kg near-infrared (IR) dye-labeled atezolizumab or TS1521, detected by LI-COR imaging. FIG. 7B show quantification of antibody persistence in the tumor over time from panel FIG. 7A Error bars represent s.d. (n=4). FIG. 7C shows bioavailability of tumor PD-L1. NSG mice bearing MDA-MB-231 tumors were injected i.v. with 1 mg/kg TS1521 for 24 or 96 hr. Surface PD-L1 that was not bound to a PD-1 competitive antibody (i.e., bioavailable PD-L1) was quantified using a radiolabeled peptide.

### DETAILED DESCRIPTION

Programmed death-ligand 1 (PD-L1) drives inhibition of antigen-specific T cell responses through engagement of its receptor programmed death-1 (PD-1), which is expressed primarily on activated T cells. Overexpression of these immune checkpoint proteins in the tumor microenvironment has motivated the design of targeted antibodies that disrupt this interaction. Despite the clinical success of these competitive antibodies, response rates remain low, necessitating novel approaches to enhance immunotherapeutic performance. Provided herein are antibody fusion proteins that can block immune checkpoint pathways through a distinct mechanism targeting molecular trafficking dynamics. By engaging multiple epitopes on PD-L1, the disclosed engineered multiparatopic antibody constructs can induce rapid clustering, internalization, and lysosomal degradation of the target protein in a topology- and epitope-dependent manner.

Provided herein are anti-PD-L1 multiparatopic antibody constructs that include a plurality of antigen-binding fragments, wherein a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope.

Also provided herein are nucleic acids that include sequences that encode polypeptides making up any one of the anti-PD-L1 multiparatopic antibody constructs described herein. Also provided herein are vectors that include any one of the nucleic acids described herein, and cells that include any one of the nucleic acids or vectors described herein. Also described herein are pharmaceutical compositions that include any one of the anti-PD-L1 multiparatopic antibody constructs, nucleic acids, vectors, or cells described herein, and kits that include any one of the pharmaceutical compositions described herein.

Also provided herein are methods of producing an anti-PD-L1 multiparatopic antibody construct that include (a) culturing any one of the cells described herein in a liquid culture medium under conditions sufficient to produce the anti-PD-L1 multiparatopic antibody construct; and (b) recovering the anti-PD-L1 multiparatopic antibody construct from the cell and/or liquid culture medium.

Also provided herein are methods of treating a disease in a subject in need thereof that include administering a therapeutically effective amount of any one of the anti-PD-L1 multiparatopic antibody constructs or any one of the pharmaceutical compositions described herein to the subject.

Various non-limiting aspects of these anti-PD-L1 multiparatopic antibody constructs are described herein, and can be used in any combination without limitation. Additional aspects of various components of methods of making and using the anti-PD-L1 multiparatopic antibody constructs are known in the art.

As used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “antibody” refers to an immunoglobulin molecule that includes one or more antigen-binding domains that specifically bind to a particular antigen. In some embodiments, the term encompasses any polypeptide or polypeptide complex that includes immunoglobulin structural elements sufficient to confer specific binding. Exemplary antibodies include, but are not limited to monoclonal antibodies, polyclonal antibodies, and fragments thereof. In some embodiments, an antibody may include one or more sequence elements are humanized, primatized, chimeric, etc., as is known in the art. In some embodiments, the term “antibody” is used to refer to one or more of the art-known or developed constructs or formats for utilizing antibody structural and functional features in alternative presentation. For example, an antibody utilized in accordance with the present disclosure can be in a format selected from, but not limited to, intact IgA, IgG, IgE, or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR, VNAR, or fragments thereof); cameloid antibodies; masked antibodies (e.g., Probodies®); Small Modular ImmunoPharmaceuticals (“SMIPs™”); single chain or Tandem diabodies (TandAb®); VHHs, Anticalins®; Nanobodies® minibodies; BiTERs; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In some embodiments, an antibody is or comprises a polypeptide whose amino acid sequence includes one or more structural elements recognized by those skilled in the art as a complementarity determining region (CDR). In some embodiments, an antibody is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments, an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments, an antibody is or comprises a polypeptide whose amino acid sequence includes structural elements recognized by those skilled in the art as an immunoglobulin variable domain. In some embodiments, an antibody is a polypeptide protein having a binding domain which is homologous or largely homologous to an immunoglobulin-binding domain.

As used herein, the term “antigen” refers to a molecule or molecular structure that binds to a specific antibody or T-cell receptor. In some embodiments, an antigen binds to an antibody or T-cell receptor and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

As used herein, an “antigen-binding fragment” or “antigen-binding domain” refers to a portion of an antibody or T-cell receptor that specifically binds to a target moiety or entity. Typically, the interaction between an antigen binding fragment and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding fragment may be or comprise a polypeptide (or complex thereof). In some embodiments, an antigen binding domain is part of a fusion polypeptide.

It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

As used herein, in general, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

As used herein, the term “epitope” refers to a portion of an antigen that specifically binds to an antigen-binding fragment. Epitopes can, for example, consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, a “vector” or “recombinant vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### Anti-PD-L1 Multiparatopic Antibody Construct

Provided herein are anti-PD-L1 multiparatopic antibody constructs that include a plurality of antigen-binding fragments, wherein a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope.

### Programmed Death-Ligand 1 (PD-L1)

Programmed death-ligand 1 (PD-L1) (also referred to as CD274 or B7-H1) is a transmembrane protein that performs a major role in suppressing the adaptive immune system during particular events such as pregnancy, tissue allografts, autoimmune disease and other disease states. PD-L1 is also expressed on the neoplastic cells of many different cancers. By binding to PD-1 on T-cells, PD-L1 expression is a major mechanism by which tumor cells can evade immune attack. PD-L1 over-expression may conceptually be due to two mechanisms, intrinsic and adaptive. Intrinsic expression of PD-L1 on cancer cells is related to cellular/genetic aberrations in these neoplastic cells. Activation of cellular signaling including the AKT and STAT pathways results in increased PD-L1 expression. In primary mediastinal B-cell lymphomas, gene fusion of the MHC class II transactivator (CIITA) with PD-L1 or PD-L2 occurs, resulting in overexpression of these proteins. Amplification of chromosome 9p23-24, where PD-L1 and PD-L2 are located, leads to increased expression of both proteins in classical Hodgkin lymphoma. Adaptive mechanisms are related to induction of PD-L1 expression in the tumor microenvironment. PD-L1 can be induced on neoplastic cells in response to interferon γ. In microsatellite instability colon cancer, PD-L1 is mainly expressed on myeloid cells in the tumors, which then suppress cytotoxic T-cell function.

In a tumor microenvironment, programmed death ligand 1 (PD-L1) can perform a vital role in tumor progression and survival by escaping tumor neutralizing immune surveillance. Enhancing T cell activation by blocking the PD-1 and PD-L1 inhibitory pathway has shown beneficial anti-tumor responses and long-term remissions in a subset of patients with a broad spectrum of cancers. Therefore, use of an inhibitor that blocks the interaction of PD-L1 with the PD-1 can help prevent PD-1 stimulation (e.g., on T cells), thereby increasing T cell function signals and immune cell responses.

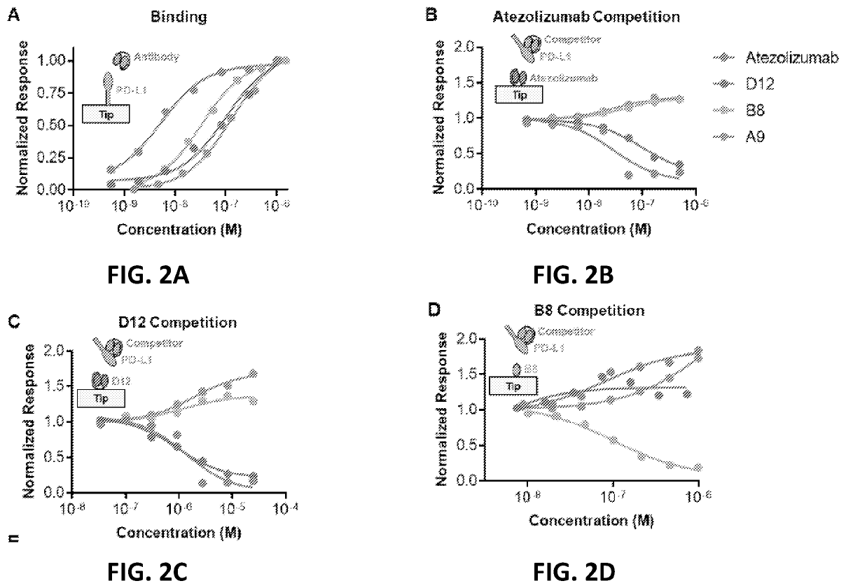

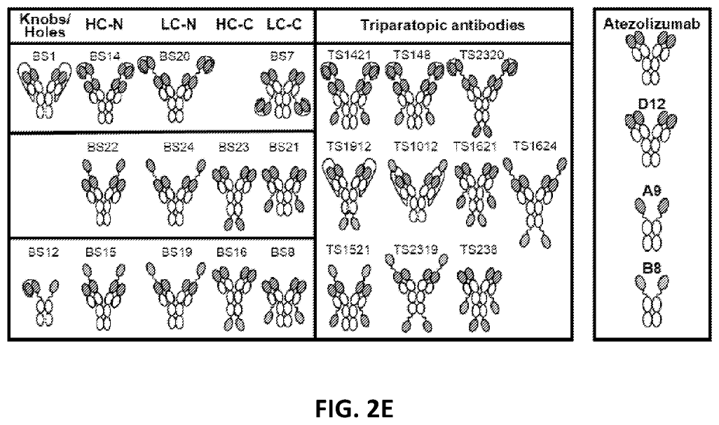

In some embodiments, a multiparatopic antibody construct can be engineered to engage multiple epitopes on PD-L1, wherein the multiparatopic antibody construct can induce rapid clustering, internalization, and lysosomal degradation of the PD-L1 protein. As used herein, a “multiparatopic antibody construct” can refer to an engineered antibody construct that comprises a plurality of distinct antigen-binding fragments that bind to the same target (e.g., PD-L1), wherein the plurality of distinct antigen-binding fragments each bind to a different epitope on the target protein. In some embodiments, an anti-PD-L1 multiparatopic antibody construct can include a plurality of antigen-binding fragments. In some embodiments, the anti-PD-L1 multiparatopic antibody construct can include two or more (e.g., two, three, four, or five) antigen-binding fragments. In some embodiments, the anti-PD-L1 multiparatopic antibody construct can be in a format (e.g., having a specific light chain, heavy chain, and/or antigen-binding fragment geometry) shown in FIG. 2E. In some embodiments, the anti-PD-L1 multiparatopic antibody construct can be in a format shown in FIG. 2E, but can include different specific antigen-binding fragments than those shown in FIG. 2E (e.g., any of the antigen-binding fragments described herein).

In some embodiments, the anti-PD-L1 multiparatopic antibody construct can include a first antigen-binding fragment and a second antigen-binding fragment of the plurality of antigen-binding fragments. In some embodiments, a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope. In some embodiments, the anti-PD-L1 multiparatopic antibody construct further comprises a third antigen-binding fragment of the plurality of antigen-binding fragments, wherein the third antigen-binding fragment binds specifically to a third PD-L1 epitope. In some embodiments, the anti-PD-L1 multiparatopic antibody construct further comprises a fourth antigen-binding fragment of the plurality of antigen-binding fragments, wherein the fourth antigen-binding fragment binds specifically to a fourth PD-L1 epitope. In some embodiments, the anti-PD-L1 multiparatopic antibody construct further comprises a fifth antigen-binding fragment of the plurality of antigen-binding fragments, wherein the fifth antigen-binding fragment binds specifically to a fifth PD-L1 epitope.

In some embodiments, the anti-PD-L1 multiparatopic antibody construct comprises an antibody. In some embodiments, the anti-PD-L1 multiparatopic antibody construct comprises a human immunoglobulin G1 (IgG) antibody. In some embodiments, the anti-PD-L1 multiparatopic antibody construct comprises an anti-PD-L1 antibody (e.g., Avelumab, Atezolizumab, Durvalumab). In some embodiments, the anti-PD-L1 multiparatopic antibody construct comprises atezolizumab. In some embodiments, the anti-PD-L1 multiparatopic antibody construct comprises (i) a first polypeptide comprising a light chain, and (ii) a second polypeptide comprising a heavy chain.

In some embodiments, the anti-PD-L1 multiparatopic antibody construct comprises a knobs-in-holes assembly. In some embodiments, the anti-PD-L1 multiparatopic antibody construct comprises a knobs-in-holes Fc mutation. In some embodiments, a first polypeptide comprises an engineered light chain constant region, and a second polypeptide comprises an engineered heavy chain constant region, wherein the engineered light chain constant region and the engineered heavy chain constant region preferentially associate with each other as compared to non-engineered light and heavy chain constant regions. In some embodiments, the engineered heavy chain constant region comprises a mutation. In some embodiments, the mutation is a N297A mutation.

### Antigen Binding Fragment

In some embodiments, the antigen-binding fragment comprises a variable domain of an antibody. In some embodiments, the antigen-binding fragment comprises a scFv. In some embodiments, the antigen-binding fragment comprises a VNAR. In some embodiments, the antigen-binding fragment comprises: cameloid antibodies; masked antibodies (e.g., Probodies®); Small Modular ImmunoPharmaceuticals (“SMIPs™”); single chain or Tandem diabodies (TandAb®); VHIIs; Anticalins®; Nanobodies® minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the anti-PD-L1 multiparatopic antibody construct comprises a plurality of antigen-binding fragments. In some embodiments, the anti-PD-L1 multiparatopic antibody construct comprises a first antigen-binding fragment that binds specifically to a first epitope of a target protein, and a second antigen-binding fragment that binds specifically to a second epitope of the target protein. In some embodiments, the anti-PD-L1 multiparatopic antibody construct further comprises a third antigen-binding fragment that binds specifically to a third epitope of the target protein. In some embodiments, the anti-PD-L1 multiparatopic antibody construct further comprises a fourth antigen-binding fragment that binds specifically to a fourth epitope of the target protein In some embodiments, the anti-PD-L1 multiparatopic antibody construct further comprises a fifth antigen-binding fragment that binds specifically to a fifth epitope of the target protein.

In some embodiments, an antigen-binding fragment of the plurality of antigen-binding fragments comprises a variable domain. In some embodiments, the antigen-binding fragment comprises a variable domain of an antibody. In some embodiments, the antigen-binding fragment comprises a variable domain that binds specifically to an epitope of a target protein. In some embodiments, the variable domain binds specifically to a PD-L1 epitope. In some embodiments, the anti-PD-L1 multiparatopic antibody construct comprises an antibody whose variable domains bind specifically to a PD-L1 epitope.

In some embodiments, an antigen-binding fragment can be conjugated to a first polypeptide (e.g., a first polypeptide that comprises a light chain or heavy chain). In some embodiments, an antigen-binding fragment can be conjugated to a second polypeptide (e.g., a second polypeptide that comprises a light chain or heavy chain). In some embodiments, an antigen-binding fragment can be N-terminally positioned relative to the light chain. In some embodiments, an antigen-binding fragment can be C-terminally positioned relative to the light chain. In some embodiments, an antigen-binding fragment can be N-terminally positioned relative to the heavy chain. In some embodiments, an antigen-binding fragment can be C-terminally positioned relative to the heavy chain.

In some embodiments, the first antigen-binding fragment and/or the second antigen-binding fragment is conjugated to the first polypeptide. In some embodiments, the first antigen-binding fragment and/or the second antigen-binding fragment is N-terminally positioned relative to the light chain. In some embodiments, the first antigen-binding fragment and/or the second antigen-binding fragment is C-terminally positioned relative to the light chain. In some embodiments, the first antigen-binding fragment and/or the second antigen-binding fragment is conjugated to the second polypeptide. In some embodiments, the first antigen-binding fragment and/or the second antigen-binding fragment is N-terminally positioned relative to the heavy chain. In some embodiments, the first antigen-binding fragment and/or the second antigen-binding fragment is C-terminally positioned relative to the heavy chain. In some embodiments, the third antigen-binding fragment is conjugated to the first polypeptide. In some embodiments, the third antigen-binding fragment is N-terminally positioned relative to the light chain. In some embodiments, the third antigen-binding fragment is C-terminally positioned relative to the light chain. In some embodiments, the third antigen-binding fragment is conjugated to the second polypeptide. In some embodiments, the third antigen-binding fragment is N-terminally positioned relative to the heavy chain. In some embodiments, the third antigen-binding fragment is C-terminally positioned relative to the heavy chain. In some embodiments, the fourth antigen-binding fragment is C-terminally positioned relative to the light chain. In some embodiments, the fourth antigen-binding fragment is conjugated to the second polypeptide. In some embodiments, the fourth antigen-binding fragment is N-terminally positioned relative to the heavy chain. In some embodiments, the fourth antigen-binding fragment is C-terminally positioned relative to the heavy chain. In some embodiments, the fifth antigen-binding fragment is C-terminally positioned relative to the light chain. In some embodiments, the fifth antigen-binding fragment is conjugated to the second polypeptide. In some embodiments, the fifth antigen-binding fragment is N-terminally positioned relative to the heavy chain. In some embodiments, the fifth antigen-binding fragment is C-terminally positioned relative to the heavy chain.

In some embodiments, an antigen-binding fragment can bind specifically to a PD-L1 epitope. In some embodiments, an antigen-binding fragment can comprise or consist of a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, an antigen-binding fragment can comprise or consist of a sequence of SEQ ID NO: 9, SEQ ID NO: 10, or SEQ ID NO: 11. In some embodiments, the first antigen-binding fragment and/or the second antigen-binding fragment comprises or consists of a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the first antigen-binding fragment and/or the second antigen-binding fragment comprises or consists of a sequence of SEQ ID NO: 9, SEQ ID NO: 10, or SEQ ID NO: 11. In some embodiments, the third antigen-binding fragment comprises a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the third antigen-binding fragment comprises or consists of a sequence of SEQ ID NO: 9, SEQ ID NO: 10, or SEQ ID NO: 11. In some embodiments, the fourth antigen-binding fragment comprises or consists of a sequence of SEQ ID NO: 9, SEQ ID NO: 10, or SEQ ID NO: 11. In some embodiments, the fifth antigen-binding fragment comprises or consists of a sequence of SEQ ID NO: 9, SEQ ID NO: 10, or SEQ ID NO: 11. In some embodiments, the plurality of antigen-binding fragments comprise or consist of two or more sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the plurality of antigen-binding fragments comprise two or more sequences selected from the group consisting of: SEQ ID NO: 9, SEQ ID NO: 10, or SEQ ID NO: 11.

### Linker Sequence

In some embodiments, one or more linkers (e.g., flexible linkers) can be introduced into the multiparatopic antibody construct to provide flexibility at one or more of the junctions between domains, between moieties, between moieties and domains, or at any other junctions where a linker would be beneficial. In some embodiments, the multiparatopic antibody construct further comprises a linker sequence. In some embodiments, the linker sequence can be a flexible linker sequence. In some embodiments, the linker sequence is a synthetic linker sequence.

In some embodiments, the anti-PD-L1 multiparatopic antibody construct further comprising a linker sequence. In some embodiments, the linker sequence can be positioned between an antigen-binding fragment and the first polypeptide. In some embodiments, the linker sequence can be positioned between an antigen-binding fragment and the second polypeptide. In some embodiments, the linker sequence is positioned between the first polypeptide and the first antigen-binding fragment and/or the second antigen-binding fragment. In some embodiments, the linker sequence is positioned between the second polypeptide and the first antigen-binding fragment and/or the second antigen-binding fragment.

In some embodiments, the linker sequence can include a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments, the linker sequence can include a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids in length.

In some embodiments, a linker sequence can be rich in glycine (Gly or G) residues. In some embodiments, the linker sequence can be rich in serine (Ser or S) residues. In some embodiments, the linker sequence can be rich in glycine and serine residues. In some embodiments, the linker sequence has one or more Gly-Gly-Gly-Gly-Ser (GGGGS or G₄S) sequences (e.g., 1, 2, 3, 4, 5, 6, 7, 8, 9, or 10 or more GGGGS sequences). In some embodiments, the linker sequence comprises a (G₄S)₃ or a (G₄S)₂ linker. In some embodiments, the linker sequence comprises a GGGGSGGGGSGGGGS (SEQ ID NO: 15). In some embodiments, the linker sequence comprises a GGGGSGGGGS (SEQ ID NO: 16).

### Methods of Producing an Anti-PD-L1 Multiparatopic Antibody Construct

### Nucleic Acids and Vectors

Provided herein are nucleic acids encoding polypeptides making up any one of the anti-PD-L1 multiparatopic antibody constructs described herein. As used herein, “nucleic acid” is used to include any compound and/or substance that comprise a polymer of nucleotides. In some embodiments, a polymer of nucleotides is referred to as polynucleotides. Exemplary nucleic acids or polynucleotides can include, but are not limited to, ribonucleic acids (RNAs) and/or deoxyribonucleic acids (DNAs).

In some embodiments, nucleic acid constructs may be inserted into a recombinant vector or viral vector by methods known to the art, and nucleic acid molecules may be operably linked to an expression control sequence. Non-limiting examples of recombinant vectors include plasmid vectors, transposon vectors, cosmid vectors, and viral vectors (e.g., any adenoviral vectors (AV), cytomegaloviral (CMV) vectors, simian viral (SV40) vectors, adeno-associated virus (AAV) vectors, lentiviral vectors, and retroviral vectors). In some embodiments, the recombinant vector is a viral vector.

Additional sequences can be added to such cloning and/or expression sequences to optimize their function in cloning and/or expression, to aid in isolation of the polynucleotide, or to improve the introduction of the polynucleotide into a cell. Use of cloning vectors, recombinant vectors, adapters, and linkers is well known in the art.

Also provided herein are recombinant vectors comprising any one the nucleic acid molecules described herein. Also provided herein are cells comprising any one of the nucleic acid molecules or the vectors described herein. In some embodiments, nucleic acid molecules are inserted into a vector that is able to express polypeptides making up any one of the anti-PD-L1 multiparatopic antibody constructs of the present disclosure when introduced into an appropriate cell. In some embodiments, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., an eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

Also provided herein are methods of producing an anti-PD-L1 multiparatopic antibody construct that include (a) culturing any one of the cells described herein in a liquid culture medium under conditions sufficient to produce the anti-PD-L1 multiparatopic antibody construct; and (b) recovering the anti-PD-L1 multiparatopic antibody construct from the cell and/or liquid culture medium. In some embodiments, the method further comprises isolating the recovered anti-PD-L1 multiparatopic antibody construct. In some embodiments, the method further comprises formulating the isolated anti-PD-L1 multiparatopic antibody construct into a pharmaceutical composition.

### Therapeutic Applications

Provided herein are pharmaceutical compositions that include any of anti-PD-L1 multiparatopic antibody constructs, the nucleic acid molecules, the vectors, or the cells described herein. In some embodiments, pharmaceutical compositions provided herein include a pharmaceutically acceptable carrier.

Also provided herein are methods of treating a disease in a subject in need thereof that include administering a therapeutically effective amount of any one of the anti-PD-L1 multiparatopic antibody constructs or any one of the pharmaceutical compositions described herein to the subject. In some embodiments, the subject is a human. Also provided herein, are uses of any one of the anti-PD-L1 multiparatopic antibody constructs or any one of the pharmaceutical compositions described herein in the manufacture of a medicament for the treatment of a disease.

In some embodiments, the disease is a cancer. In some embodiments, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma. In some embodiments, the cancer is a bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, prostate cancer, or combinations thereof.

In some embodiments, the present disclosure provides pharmaceutical compositions that include any of anti-PD-L1 multiparatopic antibody constructs, the nucleic acid molecules, the vectors, or the cells described herein, and a pharmaceutically acceptable carrier. In some embodiments, a pharmaceutical composition can include a buffer, a diluent, solubilizer, emulsifier, preservative, adjuvant, an excipient, or any combination thereof. In some embodiments, a composition, if desired, can also contain one or more additional therapeutically active substances.

In some embodiments, compositions are formulated for parenteral administration. For example, a pharmaceutical composition provided herein may be provided in a sterile injectable form (e.g., a form that is suitable for subcutaneous injection, hepatic artery infusion, or intravenous infusion). For example, in some embodiments, a pharmaceutical composition is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

In some embodiments, a pharmaceutical composition of the present disclosure is formulated with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. A vehicle or lyophilized powder can contain additives that maintain isotonicity (e.g., sodium chloride, mannitol) and chemical stability (e.g., buffers and preservatives). In some embodiments, a formulation is sterilized by known or suitable techniques. A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening, or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

### EXAMPLES

The disclosure is further described in the following examples, which do not limit the scope of the disclosure described in the claims.

### Example 1—Discovery of Novel PD-L1-Targeted Antibodies

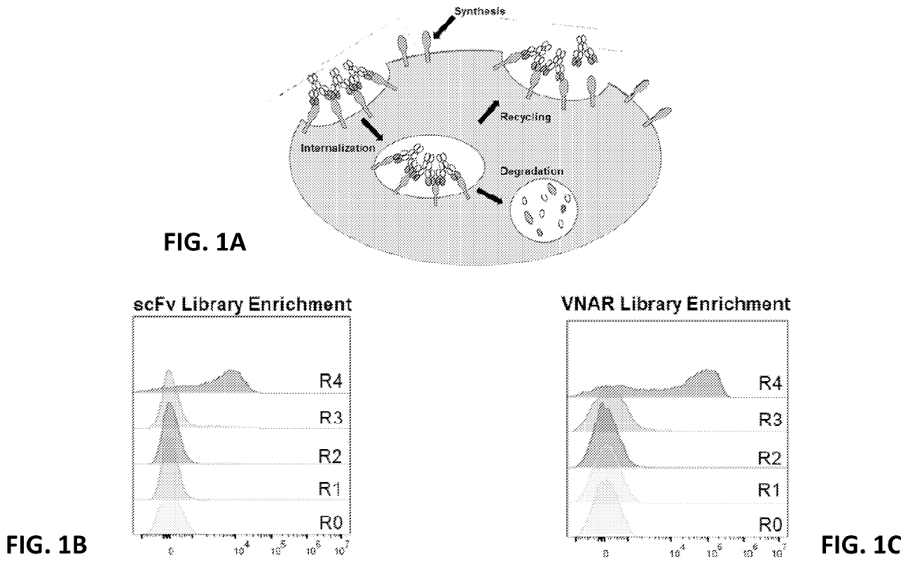

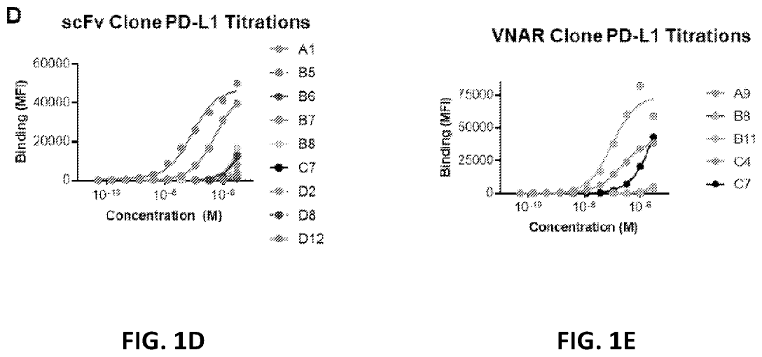

To identify new PD-L1 binding proteins, a naive yeast-displayed synthetic antibody single-chain variable fragment (scFv) library was selected against the extracellular domain (ECD) of PD-L1 by performing iterative rounds of magnetic-activated cell sorting (MACS) and fluorescence activated cell sorting (FACS) (FIG. 1B). Several PD-L1-specific clones were identified from the enriched library, with affinities ranging from 10- to >1,000-fold weaker than atezolizumab (FIG. 1C, Tables 1, 2). Additionally, a yeast-displayed variable new antigen receptor (VNAR) nanobody library developed through immunization of nurse sharks with the PD-L1 ECD was screened. Successive rounds of MACS and FACS led to identification of additional PD-L1 binding domains, which also showed a range of affinities from 10- to >1,000-fold weaker than atezolizumab (FIGS. 1D-1E, Tables 1, 2).

TABLE 1
Protein sequences of anti-PD-L1 clones

| | | SEQ ID |
|---|---|---|
| Clone | Amino Acid Sequence | NO: |
| A1 scFv | DIQMTQSPSSLSASVGDRVTITCRASQSISSYLNWYQQKPGKAPKLLIYA | 1 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARVDAMYYPYFDYWGQGTLVTVS | |
| | S | |
| B5 scFv | EIVLTQSPGTLSLSPGERATLSCRASQSVSSSYLAWYQQKPGQAPRLLIY | 2 |
| | GASSRATGIPDRFSGSGSGTDFTLTISRLEPEDFAVYYCQQYGSSPSTFGP | |
| | GTKVDIKSGILGTTAASGSSGGSSSGAEVQLVQSGAEVKKPGESLKISCK | |
| | GSGYSFTSYWIGWVRQMPGKGLEWMGIIYPGDSDTRYSPSFQGQVTIS | |
| | ADKSISTAYLQWSSLKASDTAVYYCARYDSGGLYYYDFDYWGQGTLV | |
| | TVSS | |
| B6 scFv | DIQMTQSPSSLSASVGDRVTITCRASQSISSYLNWYQQKPGKAPKLLIYA | 3 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARNYELDYFDYWGQGTLVTVSS | |
| B7 scFv | DIQMTQSPSSLSASVGDRVTITCRASQSISSYLNWYQQKPGKAPKLLIYA | 4 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARYDYGSDSPLHRSFDYWGQGTLV | |
| | TVSS | |
| B8 scFv | DIQMTQSPSSLSASVGDRVTITCRASQSISSYLNWYQQKPGKAPKLLIYA | 5 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARDGYYSDFDYWGQGTLVTVSS | |
| C7 scFv | DIQMTQSPSSLSASVGDRVTITCRASQSISSYLNWYQQKPGKAPKLLIYA | 6 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARYDYGSDAGLTYFDYWGQGTLV | |
| | TVSS | |
| D2 scFv | EIVLTQSPGTLSASVGDRVTITCRASQSISSYLNWYQQKPGKAPKLLIYA | 7 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARDYLYSDFDYWGQGTLVTVSS | |
| D8 scFv | DIQMTQSPSSLSASVGDRVTITCRASQSISSYLNWYQQKPGKAPKLLIYA | 8 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARGSGYSGYTAADDEDYWGQGTL | |
| | VTVSS | |
| D12 scFv | DIQMTQSPSSLSASVGDRVTITCRASQSISSYLNWYQQKPGKAPKLLIYA | 9 |
| | ASSLQSGVPSRFSGSGSGTDFTLTISSLQPEDFATYYCQQSYSTPLTFGQG | |
| | TKVEIKSGILGTTAASGSSGGSSSGAQVQLVQSGAEVKKPGSSVKVSCK | |
| | ASGGTFSSYAISWVRQAPGQGLEWMGGIIPIFGTANYAQKFQGRVTITA | |
| | DKSTSTAYMELSSLRSEDTAVYYCARYYDLYDSYPEYFDYWGQGTLV | |
| | TVSS | |
| A9 | ARVDQTPQTITKETGESLTINCVVRDTACALVRTFWYRKKSGSTNEESIS | 10 |
| VNAR | KGGRYVETVNRGSKSFSLRINDLTVEDSGTYRCKVFGWCRMRDVYGG | |
| | GTVVTVN | |
| B8 | ARVDQTPQTITKETGESLTINCVLRDASYALGSTCWYRKKSGSTNEESIS | 11 |
| VNAR | IGGRYVETVNSGSKSFSLRINDLTVEDSGTYRCGVDSWCPPVCSWAYA | |
| | GCGDGTAVTVN | |
| B11 | ARVDQTPQTITKETGESLTINCVLRDSNCALSSTYWYRKKSGSTNEESIS | 12 |
| VNAR | KGGRYVETVNSGSKSFSLRINDLTVEDSGTYRCRLFVSWLSDYDCATY | |
| | YVSGGGTVVTVN | |
| C4 | ARVDQTPLTITKETGESLTINCVLRDSNCALSSTYWYRKSSGSTNEEIISK | 13 |
| VNAR | GGRYVETVNSGSNSFSLRINDLTVEDSGTYRCKVFGWCRMRDVYGGG | |
| | TVVTVN | |
| C7 | ARVDQTPQTITKETGESLTINCVVRDSACALVRTFWYRKKSGSTNEESIS | 14 |
| VNAR | KGGRYVETVNSGSKSFSLRINDLTVEDSGTYRCKVFGWCRMRDVYGG | |
| | GTVVTVN | |

TABLE 2
Equilibrium K_{D} Values from Binding Studies

| | | | Equilibrium | |
|---|---|---|---|---|
| Treatment | Target | Method | K_{D} (nM) | FIG. |
| A1 (scFv) | PD-L1 | Yeast | 580 | 1C |
| B5 (scFv) | PD-L1 | Yeast | >1000 | 1C |
| B6 (scFv) | PD-L1 | Yeast | >1000 | 1C |
| B7 (scFv) | PD-L1 | Yeast | >1000 | 1C |
| B8 (scFv) | PD-L1 | Yeast | >1000 | 1C |
| C7 (scFv) | PD-L1 | Yeast | >1000 | 1C |
| D2 (scFv) | PD-L1 | Yeast | >1000 | 1C |
| D8 (scFv) | PD-L1 | Yeast | >1000 | 1C |
| D12 (scFv) | PD-L1 | Yeast | 88.6 | 1C |
| Atezolizumab (scFv) | PD-L1 | Yeast | 1.74 | 1C |
| A9 (VNAR) | PD-L1 | Yeast | 225 | 1D |
| B8 (VNAR) | PD-L1 | Yeast | 97.3 | 1D |
| B11 (VNAR) | PD-L1 | Yeast | >1000 | 1D |
| C7 (VNAR) | PD-L1 | Yeast | 400 | 1D |
| C4 (VNAR) | PD-L1 | Yeast | >1000 | 1D |
| Atezolizumab (scFv) | PD-L1 | BLI | 5.25 | 2A |
| D12 (scFv) | PD-L1 | BLI | 99.5 | 2A |
| B8 (VNAR) | PD-L1 | BLI | 33.5 | 2A |
| A9 (VNAR) | PD-L1 | BLI | 127 | 2A |
| Atezolizumab | PD-L1 | Cell | 0.080 | S6A |
| TS1521 | PD-L1 | Cell | 0.204 | S6A |

### Example 2—Newly Discovered Anti-PD-L1 Antibodies Target Distinct Epitopes Compared to Atezolizumab

Amongst the isolated clones from the enriched libraries, a single scFv (D12) and two VNARs (A9 and B8) were expressed in soluble form as variable fragments through transient transfection of human embryonic kidney (HEK) 293F cells, alongside the scFv of atezolizumab. Bio-layer interferometry studies revealed that the equilibrium binding affinity (K_{D}) for PD-L1 was 5.3 nM for the atezolizumab scFv, compared to 100 nM for D12, 130 nM for A9, and 34 nM for B8 (FIG. 2A, Table 2). It was then sought to examine overlap between the PD-L1 binding epitopes of the various binders through bio-layer interferometry-based cross-competition studies. Whereas the scFv D12 was found to compete with atezolizumab for PD-L1 binding, both VNAR domains (A9 and B8) were found to be noncompetitive with atezolizumab (FIG. 2B). A9 and B8 were further determined to be noncompetitive with D12 (FIG. 2C), as well as with one another (FIG. 2D), suggesting the presence of at least 3 unique binding epitopes amongst these clones. Upon examining competition of the various anti-PD-L1 binding domains with the PD-1 receptor, it was found that both atezolizumab and D12 directly compete with PD-1 for PD-L1 engagement whereas B8 is noncompetitive with PD-1. Interestingly, the interplay between A9 and PD-1 was more nuanced, with simultaneous but slightly attenuated binding of PD-1 to PD-L1 observed in the presence of the VNAR domain, possibly due to allosteric effects.

### Example 3—Engineered Anti-PD-L1 Multiparatopic Antibodies Downregulate PD-L1 Surface Expression on Cancer Cells

The 4 recombinantly expressed PD-L1-targeted proteins (scFvs atezolizumab and D12 and VNARs A9 and B8) were utilized to produce a panel of bi- and tri-paratopic antibody constructs. The variable fragments of D12, B8, and A9 were fused to the N- and C-termini of the heavy chain (HC) and/or light chain (LC) of the full-length atezolizumab human immunoglobulin G1 (IgG1) antibody using flexible (Gly4Ser)3 15 amino acid linkers (except for the C-terminal LC constructs, which used a flexible (Gly4Ser)2 10 amino acid linker). Additionally, heterodimeric antibody fusion proteins were produced using engineered knobs-into-holes mutations in the heavy chain constant domain (FIG. 2E). To ensure proper chain pairing of asymmetric multiparatopic antibodies, a method for single-chain Fab expression was utilized. As in the parent atezolizumab antibody, the N297A mutation was included in the heavy chain constant domain to reduce interactions with Fcγ receptors and thereby mitigate antibody-dependent destruction of PD-L1-expressing T cells. Atezolizumab-based multispecific antibody fusion proteins were expressed via transient transfection of HEK 293F cells, and high purity was achieved for the various antibody constructs following separation by size-exclusion chromatography.

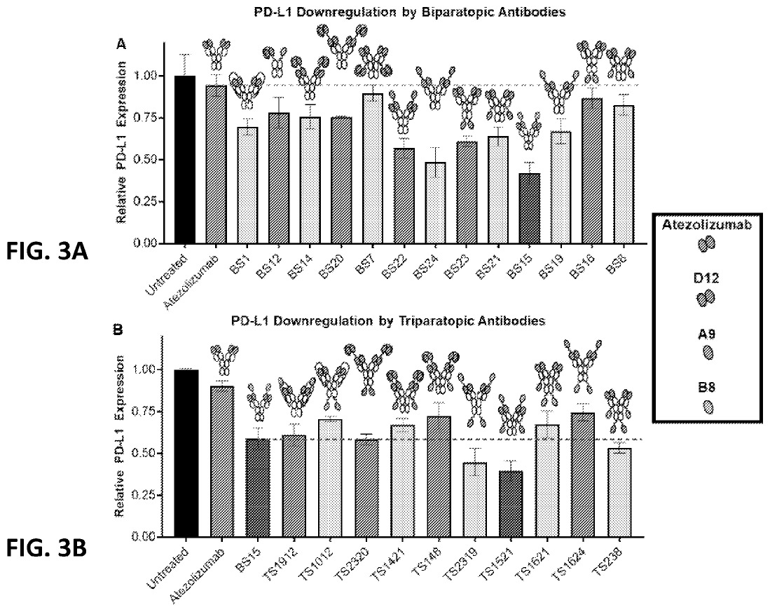

Using MDA-MB-231 human triple negative breast cancer cells, the ability of each of the engineered bi-/tri-paratopic constructs to downregulate PD-L1 expression (FIGS. 3A-3B) was examined. After treatment with antibody for 14 hours, surface PD-L1-bound antibody was stripped using an acidic solution prior to surface PD-L1 staining and detection by flow cytometry. The effectiveness of downregulation was observed to be affected by both the topology of the multiparatopic construct as well as the binding epitopes that were engaged. For example, in the context of biparatopic antibodies (FIG. 3A), fusing B8 to the N-terminus of the HC (BS15) resulted in approximately 25% greater PD-L1 downregulation compared to fusion to the N-terminus of the LC (BS19), whereas A9 and D12 did not show much variation between N-terminal HC or LC fusion orientations. Surprisingly, bivalent biparatopic antibodies (BS1 and BS12) could achieve equivalent downregulation to tetravalent constructs, although the relative performances were also dependent on geometry. D12 fusions were the least effective on average, which was expected based on the overlapping binding epitopes between D12 and atezolizumab. A9 fusions were the most effective on average. B8 fusions varied most significantly with topology, and the B8 N-terminal HC construct (BS15) was found to be the most effective of all biparatopic antibodies that were tested.

Triparatopic antibodies were generally more efficient downregulators compared to their component biparatopic antibodies, though only a few of the triparatopic constructs were more effective than BS15 (FIG. 3B). As with biparatopic antibodies, topology and epitope coverage were important determinants for downregulation extent. For instance, a triparatopic antibody with B8 N-terminally fused to the HC and A9 C-terminally fused to the LC (TS1521) elicited 28% greater downregulation than a similar antibody that relocated B8 to the C-terminus of the HC (TS1621) and 27% greater downregulation than another similar antibody that substituted D12 at the HC N-terminus (TS1421). Of note, only triparatopic antibodies that engaged 3 unique epitopes of PD-L1 (i.e., containing atezolizumab scFv plus the A9 and B8 VNARs) were found to be more effective than the most active biparatopic antibody (BS15). Amongst the triparatopic antibodies with 3 unique epitopes that were tested, geometries that positioned A9 at the C-terminus and B8 at the N-terminus were most effective at inducing PD-L1 downregulation. Overall, MDA-MB-231 downregulation studies identified the most actively downregulating anti-PD-L1 multiparatopic antibody in the panel to be TS1521, which decreased surface PD-L1 levels by 60-70%.

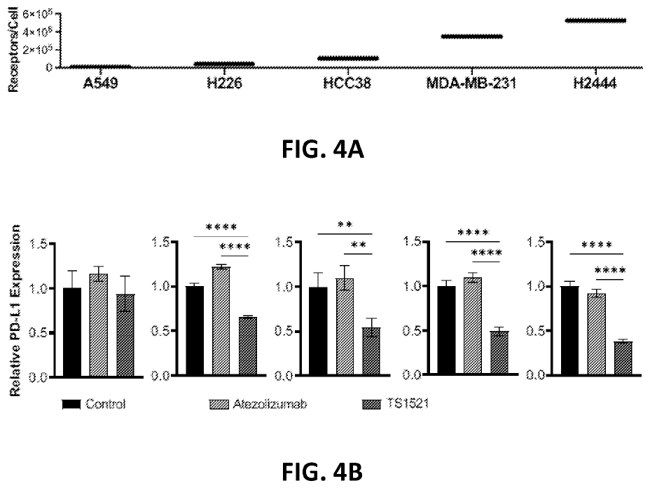

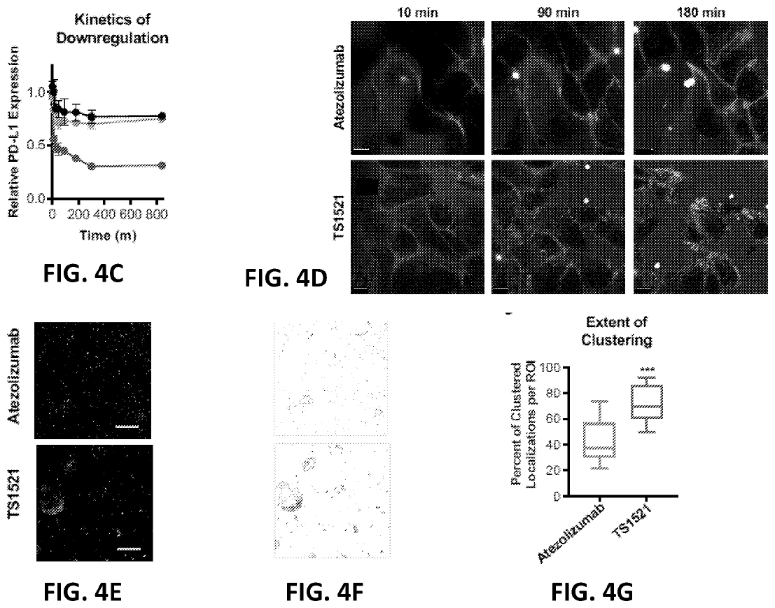

Comparison of downregulation induced by TS1521 in a range of tumor cell lines with differing levels of surface PD-L1 demonstrated that the extent of downregulation increases modestly with increasing PD-L1 expression up to a density of at least 600,000 receptors/cell (FIG. 4A-4B). Very low receptor density (for instance on the A549 cell line) eliminated multiparatopic antibody-mediated PD-L1 downregulation effect entirely, which could be due to geometric challenges in cross-linking PD-L1 when few copies are present on the surface. A similar trend of downregulation dependence on PD-L1 surface density was observed for multiparatopic antibodies BS1, BS15, and TS2319. Interestingly, the threshold for PD-L1 surface density required to enable downregulation was found to vary with antibody design (FIG. 4B). For instance, the biparatopic antibody BS15 induced significant downregulation on cells that expressed >41,000 receptors/cell, whereas the triepitopic antibody TS2319 only induced significant downregulation on cells that expressed >347,000 receptors/cell. Downregulation was also found to be dependent on antibody concentration, with maximal reduction in PD-L1 surface levels observed at saturating concentrations of multiparatopic antibody. This result is consistent with a model in which surface cross-linking of PD-L1 precipitates downregulation. Taken together, the results of the downregulation assays establish that multiparatopic antibodies mediate robust downregulation of surface PD-L1 across cancer cell lines in a manner that is dependent on antibody topology, epitope engagement, and surface protein density.

### Example 4—Multiparatopic Antibodies Induce PD-L1 Internalization, Clustering, and Lysosomal Sorting

It was next sought to understand the receptor trafficking patterns that underlie the downregulating effects of engineered multiparatopic antibodies. Kinetic studies with TS1521 on H2444 human NSCLC cells showed that antibody-induced downregulation occurs rapidly upon addition of the treatment, reaching steady state within 4-6 hr (FIG. 4C). In contrast, the parent monoclonal antibody (atezolizumab) did not alter PD-L1 surface levels relative to untreated cells. Live cell confocal microscopy imaging of H2444 cells showed that upon treatment with fluorescently labeled TS1521, significant PD-L1 clustering was observed within 3 hr, whereas no clustering was observed following treatment with fluorescently labeled atezolizumab (FIG. 4D). Visualization of PD-L1 clustering was found to correspond with the steady state kinetic downregulation profile, and internalization occurred (based on flow cytometry analysis) before detectable clustering was observed by microscopy (FIGS. 4C-4D). Super resolution (SR) microscopy studies on H2444 cells revealed clustering of labeled TS1521 on the cell membrane at single-molecule resolution (FIGS. 4E-4F). Compared to the more disperse surface distribution of labeled atezolizumab, labeled TS1521 was significantly more clustered, with a median of more than 60% of TS1521 molecules occurring in clusters of 10 or more, compared to less than 40% for atezolizumab (FIG. 4G).

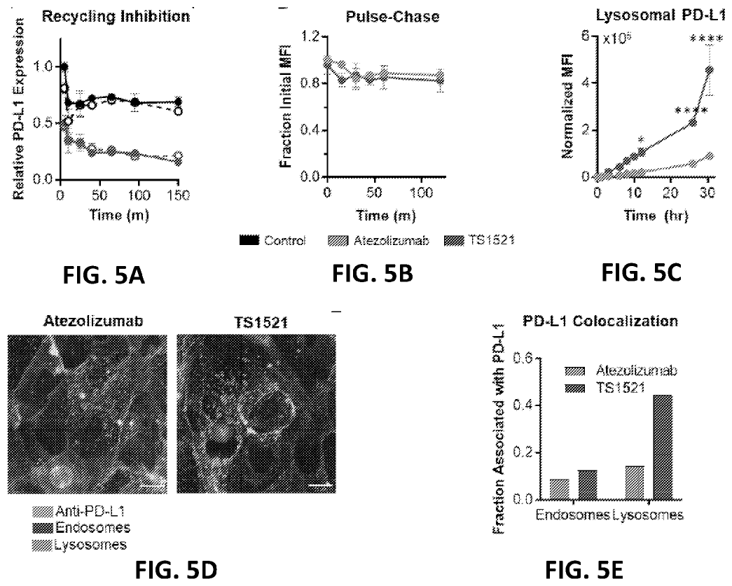

As shown in FIG. 1A, surface levels of PD-L1 are dictated by the balance between synthesis, internalization, recycling, and degradation of the protein. To determine whether PD-L1 recycling was impacted by multiparatopic antibody treatment, H2444 cells were incubated with TS1521 in the absence or presence of the recycling inhibitor monensin. No change in TS21-mediated PD-L1 downregulation was observed with or without monensin, suggesting that no recycling was occurring in the presence of the multiparatopic antibody (FIG. 5A). Interestingly, there was no difference in PD-L1 surface levels in untreated cells incubated with or without monensin, indicating that recycling was not contributing significantly to the molecular dynamics of this system. It was further confirmed that recycling was not impacted by multiparatopic antibody treatment using a pulse-chase quenching assay. In brief, H2444 cells were pulsed with Alexa Fluor 488-labeled antibody (either atezolizumab or TS1521) at 37° C. for 2 hours to allow for internalization, and then briefly incubated with an anti-Alexa Fluor 488 antibody at 4° C. to quench surface staining of PD-L1 while also preventing further internalization. Cells were subsequently returned to 37° C. for a chase period in the continued presence of the quenching antibody, so that a subsequent drop in fluorescent signal would be observed if recycling was occurring. The pulse-chase assay showed that minimal recycling of PD-L1 occurred in cells treated with either atezolizumab or TS1521 (FIG. 5B), consistent with the findings of monensin studies. Minimal recycling of PD-L1 was also observed following treatment with BS1. Notably, a less effective multiparatopic downregulating antibody (BS15) was found to enable PD-L1 recycling, perhaps contributing to its inferior efficacy in reducing surface protein levels. Divergence in PD-L1 recycling in response to treatment with TS1521 versus BS15 further highlights the relevance of topology and epitope engagement in determining how an antibody will influence molecular trafficking.

To delineate the ultimate fate of internalized PD-L1, the monoclonal antibody atezolizumab and the multiparatopic antibody TS1521 were separately conjugated to a pH-sensitive fluorescent dye, which increases in intensity at lower pH ranges characteristic of endosomal and lysosomal compartments. Fluorescence intensity was dramatically higher in H2444 cells treated with TS1521 compared to those treated with atezolizumab over the course of 36 hours, indicative of higher endosomal and lysosomal accumulation of PD-L1 in the context of the multiparatopic antibody (FIG. 5C). Significant divergence between lysosomal accumulation of the monoclonal versus the multiparatopic antibody was observed within 12 hr of treatment. Other multiparatopic antibodies (BS1 and BS15) also induced increased lysosomal trafficking relative to atezolizumab, and the extent of lysosomal accumulation corresponded to downregulation efficiency of the respective antibodies (FIGS. 3A-3B). Confocal microscopy was performed to visualize subcellular localization of fluorescently-labeled anti-PD-L1 monoclonal or multiparatopic antibodies after a 14 hour incubation with H2444 cells (FIG. 5D). Atezolizumab treatment resulted in a diffuse cell surface distribution of PD-L1, displaying minimal association of the antibody with endosomal marker Early Endosome Antigen 1 (EEA1) or lysosomal marker lysosome-associated membrane glycoprotein 1 (LAMP1). In contrast, TS1521 treatment resulted in extensive PD-L1 clustering across focal plans, with substantial evidence of both endosomal and lysosomal colocalization of the antibody. Image quantification reinforced the observed enhancement of endosomal and lysosomal trafficking of PDL1, and the fraction of lysosomes associated with PD-L1 was nearly 3-fold higher after treatment with TS1521 compared to treatment with atezolizumab (FIG. 5E). Taken together, trafficking studies established that PD-L1-targeted multiparatopic antibodies orchestrate downregulation by enhancing PD-L1 endocytosis and lysosomal sorting, with recycling playing a minimal role in regulating surface protein levels.

### Example 5—PD-L1-Targeted Multiparatopic Antibody Treatment Leads to Enhanced Immune Cell Activity

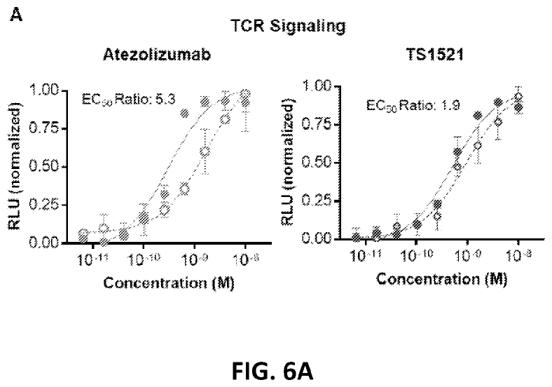

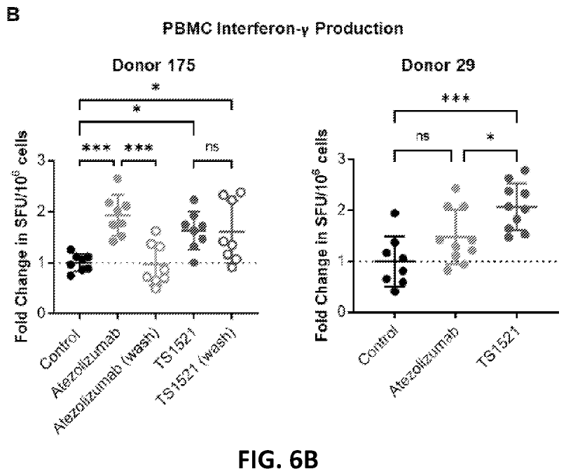

Based on the observation that engineered multiparatopic antibodies downregulate PD-L1, it was sought to determine whether this feature could be exploited to inhibit the immunosuppressive activities of the PD-1/PD-L1 pathway. As an initial test of the immunostimulatory capacity and consequent immunotherapeutic potential for multiparatopic antibodies, the performance of the monoclonal antibody atezolizumab and the multiparatopic antibody TS1521 were compared in a commercial T cell receptor (TCR) activation assay acquired from Promega™. In brief, the assay consists of 2 cell lines: Chinese hamster ovary (CHO)-K1 cells that stably express PD-L1 and a cell surface protein which elicits antigen-specific TCR activation (serving as antigen-presenting cells); and Jurkat human T cells with cognate TCRs that stably express PD-1 and an NFAT-inducible luciferase reporter (serving as effector cells). The antigen-presenting cells were first incubated with either atezolizumab or TS1521 for 2 hours to allow for PD-L1 downregulation. In some samples, the antibody was then removed, followed by addition of PD-1-expressing effector cells, whereas in other samples the antibody was still present after the addition of effector cells. NFAT-induced luminescence was used as a readout for TCR activation enabled by antibody-mediated blockade of PD-1/PD-L1 signaling. In samples wherein the antibody was continuously present, TS1521 was slightly less potent in eliciting TCR activation compared to atezolizumab (FC₅₀=0.51 vs 0.30 nM) (FIG. 6A), which parallels the PD-L1 binding affinity data (Table 2). However, when the antibody was removed prior to addition of the effector cells, TS1521 led to more potent activation of TCR compared to atezolizumab (0.95 vs 1.6 nM). The more dramatic reduction in TCR activation potency for atezolizumab versus TS1521 in the continued presence versus removal of antibody (5.3-fold versus 1.9-fold reduction) suggests that the downregulation effect of the multiparatopic antibody bolsters the durability of PD-1/PD-L1 blockade.

It was then determined whether the improvement in immune effector cell activation for multiparatopic versus monoclonal antibodies would extend to a primary cell system. To this end, human peripheral blood mononuclear cells (PBMCs) isolated from donors with chronic hepatitis C virus (HCV) infection from time points at which high T cell PD-1 expression was measured were treated with either atezolizumab or TS1521 for 1 hour to allow for PD-L1 downregulation. The antibodies were then either retained or washed off. PBMCs were then stimulated with HCV-derived peptides and ELISpot assays were performed to identify the number of IFN-γ-producing cells as a measurement of immune effector cell activation (FIG. 6B). In donor 175, an increase in the number of IFN-γ-secreting cells was observed following continuous treatment with either atezolizumab or TS1521. However, in samples where the antibody was removed prior to stimulation, atezolizumab-treated cells returned to baseline levels of stimulation. In contrast, TS1521-treated samples maintained elevated levels of effector cell activity even when the antibody was removed. As in the commercial TCR activation assay, the reduced sensitivity to antibody removal for TS1521 versus atezolizumab indicates that the engineered multiparatopic antibody enhances immune checkpoint blockade through downregulation of PD-L1. Studies on a second donor corroborated the observed increase in the number of IFN-g-secreting effector cells upon treatment with either atezolizumab or TS1521, and the increase was more pronounced for the multiparatopic versus the monoclonal antibody. Collectively, immune cell signaling assays on both immortalized cell lines and primary human cells illustrate the potential for the novel strategy to enhance immune checkpoint blockade by recruiting a trafficking-focused mechanism in addition to competitive inhibition.

### Example 6—Multiparatopic Antibodies Exhibit Robust Tumor Localization and Durably Attenuate PD-L1 Availability in Mouse Cancer Models

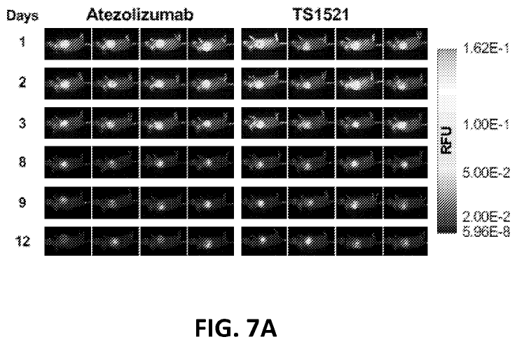

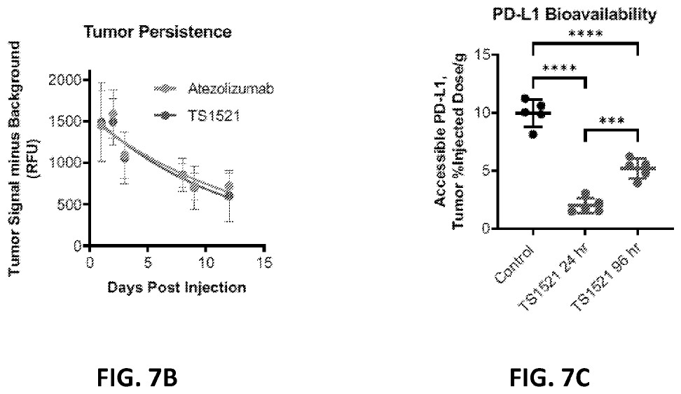

Building on the immunostimulatory effects of the PD-L1-targeted multiparatopic antibody in cellular studies, it was sought to examine the biodistribution and pharmacokinetic properties of TS1521 in a mouse tumor xenograft model. Tumor trafficking of systemically (i.v.) injected atezolizumab and TS1521 was compared in non-obese diabetic scid gamma (NSG) mice bearing MDMBA-231 tumors via near infrared imaging (FIGS. 7A-7B). No major differences in the specificity or persistence of tumor localization were observed over time between the antibodies, affirming that the engineered multiparatopic antibody recapitulates the biodistribution and pharmacokinetic properties of the clinical anti-PD-L1 monoclonal antibody. Finally, it was tested whether the multiparatopic antibody could reduce the bio-availability of PD-L1 in a mouse tumor model. To do this, a recently developed approach was leveraged, where the approach detects cell surface PD-L1 (using radiolabeled peptide, [¹⁸F]DK222) that is not bound to a PD-1-competitive antibody and therefore available for inhibitory signaling. At time points of 1- and 4-days following a single antibody injection, availability of PD-L1 was found to be significantly decreased in animals treated with TS1521 compared to untreated control animals (FIG. 7C). Although PD-L1 availability increased on day 4 compared to day 1, significant reduction was maintained compared to untreated control mice, confirming the durable effects of the engineered multiparatopic antibody in suppressing the PD-1/PD-L1 pathway. PD-L1 availability after treatment with TS1521 was similar to treatment with the clinically approved antibody atezolizumab, despite the weaker affinity of TS1521 compared to atezolizumab, highlighting the potential for sustained suppression of PD-L1 by multiparatopic antibodies.

What is claimed is:

1. An anti-PD-L1 multiparatopic antibody construct comprising:
a plurality of antigen-binding fragments,
wherein a first antigen-binding fragment of the plurality of antigen-binding fragments binds specifically to a first PD-L1 epitope, and wherein a second antigen-binding fragment of the plurality of antigen-binding fragments binds specifically to a second PD-L1 epitope.

2. The anti-PD-L1 multiparatopic antibody construct of claim 1, wherein an antigen-binding fragment of the plurality of antigen-binding fragments comprises a variable domain, wherein the variable domain binds specifically to a third PD-L1 epitope.

3. The anti-PD-L1 multiparatopic antibody construct of claim 1 or 2, wherein the antibody construct comprises a human immunoglobulin G1 (IgG) antibody.

4. The anti-PD-L1 multiparatopic antibody construct of claim 3, wherein the human IgG antibody comprises atezolizumab.

5. The anti-PD-L1 multiparatopic antibody construct of any one of claims 1-4, wherein the antibody construct further comprises (i) a first polypeptide comprising a light chain, and (ii) a second polypeptide comprising a heavy chain.

6. The anti-PD-L1 multiparatopic antibody construct of claim 5, wherein the first antigen-binding fragment and/or the second antigen-binding fragment is conjugated to the first polypeptide.

7. The anti-PD-L1 multiparatopic antibody construct of claim 5 or 6, wherein the first antigen-binding fragment and/or the second antigen-binding fragment is N-terminally positioned relative to the light chain.

8. The anti-PD-L1 multiparatopic antibody construct of any one of claims 5-7, wherein the first antigen-binding fragment and/or the second antigen-binding fragment is C-terminally positioned relative to the light chain.

9. The anti-PD-L1 multiparatopic antibody construct of any one of claims 5-8, wherein the first antigen-binding fragment and/or the second antigen-binding fragment is conjugated to the second polypeptide.

10. The anti-PD-L1 multiparatopic antibody construct of any one of claims 5-9, wherein the first antigen-binding fragment and/or the second antigen-binding fragment is N-terminally positioned relative to the heavy chain.

11. The anti-PD-L1 multiparatopic antibody construct of any one of claims 5-10, wherein the first antigen-binding fragment and/or the second antigen-binding fragment is C-terminally positioned relative to the heavy chain.

12. The anti-PD-L1 multiparatopic antibody construct of any one of claims 5-11, wherein the first polypeptide comprises an engineered light chain constant region, and wherein the second polypeptide comprises an engineered heavy chain constant region, wherein the engineered light chain constant region and the engineered heavy chain constant region preferentially associate with each other as compared to non-engineered light and heavy chain constant regions.

13. The anti-PD-L1 multiparatopic antibody construct of claim 12, wherein the engineered heavy chain constant region comprises a mutation.

14. The anti-PD-L1 multiparatopic antibody construct of claim 13, wherein the mutation is a N297A mutation.

15. The anti-PD-L1 multiparatopic antibody construct of any one of claims 1-14, further comprising a linker sequence.

16. The anti-PD-L1 multiparatopic antibody construct of claim 15, wherein the linker sequence comprises a (G₄S)₃ or a (G₄S)₂ linker.

17. The anti-PD-L1 multiparatopic antibody construct of claim 15 or 16, wherein the linker sequence is positioned between the first polypeptide and the first antigen-binding fragment and/or the second antigen-binding fragment.

18. The anti-PD-L1 multiparatopic antibody construct of any one of claims 15-17, wherein the linker sequence is positioned between the second polypeptide and the first antigen-binding fragment and/or the second antigen-binding fragment.

19. The anti-PD-L1 multiparatopic antibody construct of any one of claims 1-18, wherein the first antigen-binding fragment and/or the second antigen-binding fragment comprises a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

20. The anti-PD-L1 multiparatopic antibody construct of claim 19, wherein the first antigen-binding fragment and/or the second antigen-binding fragment comprises a sequence of SEQ ID NO: 9, SEQ ID NO: 10, or SEQ ID NO: 11.

21. The anti-PD-L1 multiparatopic antibody construct of any one of claims 1-20, wherein the plurality of antigen-binding fragments comprise two or more sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

22. The anti-PD-L1 multiparatopic antibody construct of claim 21, wherein the plurality of antigen-binding fragments comprise two or more sequences selected from the group consisting of: SEQ ID NO: 9, SEQ ID NO: 10, or SEQ ID NO: 11.

23. The anti-PD-L1 multiparatopic antibody construct of any one of claims 1-22, wherein the antigen-binding fragment comprises an antibody.

24. The anti-PD-L1 multiparatopic antibody construct of claim 23, wherein the antigen-binding fragment comprises a scFv.

25. The anti-PD-L1 multiparatopic antibody construct of claim 23, wherein the antigen-binding fragment comprises a VNAR.

26. A nucleic acid comprising sequences that encode polypeptides making up the anti-PD-L1 multiparatopic antibody construct of any one of claims 1-25.

27. A vector comprising the nucleic acid of claim 26.

28. A cell comprising the nucleic acid of claim 26 or the vector of claim 27.

29. A pharmaceutical composition comprising:
any of the anti-PD-L1 multiparatopic antibody constructs of claims 1-25, the nucleic acid of claim 26, the vector of claim 27, or the cell of claim 28; and
a pharmaceutically acceptable carrier.

30. A kit comprising the pharmaceutical composition of claim 29.

31. A method of producing an anti-PD-L1 multiparatopic antibody construct, the method comprising:
(a) culturing the cell of claim 28 in a liquid culture medium under conditions sufficient to produce the anti-PD-L1 multiparatopic antibody construct; and
(b) recovering the anti-PD-L1 multiparatopic antibody construct from the cell and/or liquid culture medium.

32. The method of claim 31, further comprising isolating the recovered anti-PD-L1 multiparatopic antibody construct.

33. The method of claim 32, further comprising formulating the isolated anti-PD-L1 multiparatopic antibody construct into a pharmaceutical composition.

34. A method of treating a disease in a subject in need thereof, the method comprising:
administering a therapeutically effective amount of the anti-PD-L1 multiparatopic antibody construct of any one of claims 1-25 or the pharmaceutical composition of claim 29 to the subject.

35. The method of claim 34, wherein the subject is a human.

36. The method of claim 34 or 35, wherein the disease is a cancer.

37. The method of claim 36, wherein the cancer is a bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, prostate cancer, or combinations thereof.
